(19) United States
(12) Patent Application Publication
(10) Pub. No.: US 2003/0036524 A1
(43) Pub. Date: Feb. 20, 2003

(54) Method of sensitising endothelial cells to prodrugs

(76) Inventor: Jens Licthenberg, Graested (DK)

Correspondence Address: BIRCH STEWART KOLASCH & BIRCH, PO BOX 747, FALLS CHURCH, VA, 22040-0747, US

(21) Appl. No.: 10/220,151

(22) PCT Filed: Mar. 8, 2001
(86) PCT No.: PCT/DK01/00152

(30) Foreign Application Priority Data
Mar. 8, 2000 (DK) PA 2000 00380

(51) Int. Cl. A61K 48/00; C12N 15/869

(52) U.S. Cl. 514/44; 424/93.2; 435/456; 435/320.1

(57) ABSTRACT

This invention relates to anti-cancer therapy. More specifically the invention relates to methods of sensitising endothelial cells to prodrugs, to recombinant viral vectors, and to methods of inhibiting angiogenesis of tumors.

### TECHNICAL FIELD

This invention relates to anti-cancer therapy. More. specifically the invention relates to methods of sensitising endothelial cells to prodrugs, to recombinant viral vectors, and to methods of inhibiting angiogenesis of tumours.

### BACKGROUND ART

Worldwide each year close to six million deaths are caused by cancer. About half of the patients stricken with cancer can be cured by surgery or radiation therapy because the tumour is localised to the site of origin. The remaining cancers include hematological malignancies and tumours that have metastasised, From these cancer types, at present only a small fraction (5-10%) can be cured by chemotherapy, the remainder are either resistant towards chemotherapy or acquire resistance during the course of therapy.

Angiogenesis is a multi-step process leading to the formation of new vessels by“sprouting” from pre-existing vessels. Angiogenesis participates in numerous physiological events, both normal and pathological. Under normal conditions, angiogenesis is associated with wound healing, corpus luteum formation and embryonic development. However, angiogenesis also has a critical role in various pathological conditions such as solid tumour growth, metastases, diabetic retinopathy, psoriasis and inflammation-related diseases such as rheumatoid arthritis. In particular, the expansion of solid tumour beyond a minimal size is critically dependent on the formation of new blood vessels to supply oxygen, nutrients and growth factors, but angiogenesis is also crucial for the formation of metastases at secondary sites.

In early studies it was found that tumours implanted into isolated perfused organs failed to grow beyond a few millimetres in diameter. However, when reimplanted into donor mice, tumours grew rapidly, beyond 1 cm³, and killed their host. In mice, the tumours became vascularized; in the perfused organs they did not. Based of these studies, a hypothesis was formulated that“solid tumours are angiogenesis-dependent,” and that“anti-angiogenesis could be a therapeutic approach in fighting cancer”.

Angiogenesis can generally be divided into three phases: First, angiogenic stimulators such as basic fibroblast growth factor (bFGF) and vascular endothelial growth factor (VEGF) are released from the tumour cells. Second, these angiogenic signals trigger proliferation of endothelial cells, secretion of proteolytic enzymes and extra cellular matrix (ECM) molecules as well as altered expression of adhesion molecules resulting in a vascular invasion of the ECM and the tumour. Third, the vascular sprouts begin to mature by forming luminal structures and fuse into loops, thereby facilitating blood circulation in the new vessel.

Under normal physiological conditions vascular, endothelial cells form a mono-layer covering approximately 1000 m² in a 70 kg person. These cells have a mean turn over of a hundred days or more. However, during angiogenesis in a neovascularized tumour endothelial cells can proliferate as fast as bone marrow progenitor cells (e.g., with a turnover time in the range of a few days).

If a tumour's vascular system could be destroyed the persistent lack of oxygen and nourishment would be expected to starve and eventually kill the tumour cells.

In this respect it has been suggested that expression systems where a gene is attached to an appropriate regulatory element targeted specifically to endothelial cells would allow for specific delivery of therapeutic agents to the endothelium. Promoter elements controlling endothelial-specific gene expression have been described.

Thus WO 97/17359 describes a novel promoter for the VEGF receptor, and WO 98/24892 describes a novel promoter for the VE-cadherin receptor. Other promoters are known in the art.

However, the expression systems suggested in the prior art for combating tumour cells are all directed to expression of toxic substances, and a number of toxic proteins of bacterial, plant or animal origin have been suggested, including the Pseudomonas exotoxin A, the Diphtheria toxin, and the tumour necrosis factor-α (see e.g. WO 97/17359, pages 24-25).

Finally, it shall be noted that methods for sensitising cells to prodrugs have been described. Thus WO 99/39740 discloses a method using lipid-mediated gene and compound delivery. However, methods of sensitising cells that target only endothelial cells have never been described.

### SUMMARY OF THE INVENTION

In contrast to the expression systems of the prior art, the present invention is devoted to the provision of an even more safe expression system. Rather than expressing toxic substances like the ones mentioned above, with the risk that these toxins become expressed at unwanted places, it is an object of the present invention to provide means for targeting proliferating endothelial cells for treating growing solid tumours and the growth of metastases that express only harmless substances and only at these particular sites.

According to the present invention tumours and metastases are combated in a two-step procedure comprising treatment only of endothelial cells with a genetically modified virus capable of incorporating a gene encoding a harmless drug-metabolising enzyme, and subsequent treatment with a non-toxic prodrug, which becomes converted to a cytotoxic agent in the endothelial cells only.

By this procedure the tumour-associated endothelial cells are destroyed and the tumour cells kept in a dormant state or killed. The method provides effective inhibition of re-growth of micro-metastases or residual tumour tissue.

The method of the present invention not only is avoid of the severe systemic side effects (toxicity) observed in connection with conventional chemotherapy, but also avoid expression of toxic substances within endothelial cells. Toxic substances are not produced until a prodrug is administered. Moreover, toxic substances are only produced in the endothelial cells. The method of the present invention therefore adds a further safety level to the methods known for combating tumours.

Accordingly, in its first aspect the invention provides a method of sensitising an endothelial cell to a prodrug, which method comprises the steps of

(i) transfecting said endothelial cell with a polynucleotide that comprises, in an operable combination,

(a) a prodrug metabolising gene, i.e. a nucleotide sequence encoding an enzyme that promotes the conversion of said prodrug into a (cytotoxic) drug; and

(b) a promoter capable of selectively promoting the transcription (expression) of the prodrug metabolising gene in said endothelial cell; and

(ii) delivering said prodrug to said endothelial cell;

wherein said endothelial cell is more sensitive to said (cytotoxic) drug than to said prodrug.

In another aspect the invention provides a recombinant viral vector comprising, in an operable combination,

a promoter and a prodrug metabolising gene,

wherein the promoter is capable of selectively promoting the transcription (expression) of the prodrug metabolising gene in an endothelial cell.

In a third aspect the invention provides a method of inhibiting tumour-induced angiogenesis in a subject, which method comprises the subsequent steps of

(i) introducing to said subject a recombinant viral vector capable of transducing endothelial cells,

which vector comprises, in an operable combination,

(a) a prodrug metabolising gene, i.e. a nucleotide sequence encoding an enzyme that promotes the conversion of a prodrug into a (cytotoxic) drug; and

(b) a promoter capable of selectively promoting the transcription (expression) of the prodrug metabolising gene in an endothelial cell; and

(ii) introducing said prodrug to said subject;

wherein said endothelial cell is more sensitive to said (cytotoxic) drug than to said prodrug.

Other objects of the invention will be apparent to the person skilled in the art from the following detailed description and examples.

### DETAILED DISCLOSURE OF THE INVENTION

Methods of Sensitising Cells

In its first aspect the present invention provides a method of sensitising an endothelial cell to a prodrug, which method comprises the steps of

(i) transfecting said endothelial cell with a polynucleotide (vector) that comprises, in an operable combination,

(a) a prodrug metabolising gene, i.e. a nucleotide sequence encoding an enzyme that promotes the conversion of said prodrug into a (cytotoxic) drug; and

(b) a promoter capable of selectively promoting the transcription (expression) of the prodrug metabolising gene in said endothelial cell; and

(ii) delivering said prodrug to said endothelial cell;

wherein said endothelial cell is more sensitive to said (cytotoxic) drug than to said prodrug.

By the term“operable combination” is meant that the prodrug metabolising gene and the promoter are connected in such a way as to permit expression of the encoded enzyme. The orientation or placement of the elements of the vector is not strict as long as the operable linkage requirement is fulfilled for control and expression of the coding polynucleotide.

As defined herein“sensitising” refers to the ability to increase the sensitivity of the cell and making it more responsive to a chemical compound (i.e. the prodrug), to which compound it previously was not sensitive, or was less sensitive to. In particular“sensitising” includes increasing the sensitivity of a cell such that exposure to a previously non-killing substance results in cell death.

In a preferred embodiment the prodrug is ifosfamide [see Keizer H J et al.: Ifosfamide treatment as a 10-day continuous intravenous infusion; J. Cancer Res. Clin. Oncol. 1995 121 297-302], and the prodrug metabolising gene is CYP 2B1 [see Kedzie K M et al.: Molecular basis for a functionally unique cytochrome P450IIB1 variant; J. Biol. Chem. 1991 266 22515-22521; and Fuji-Kuriyama Y et al.: Primary structure of a cytochrome P450: coding nucleotide sequence of phenobarbital-inducible cytochrome P450 cDNA from rat liver; Proc. Natl. Acad. Sci. USA 1982 79 2793-2797]. Upon administration of the non-toxic prodrug ifosfamide, the CYP 2B1 enzyme expressed by the endothelial cells is capable of metabolising ifosfamide to cytotoxic metabolites. The metabolites phosphoramide mustard, that alkylates DNA, and acrolein, that alkylates proteins, reach high concentrations at tumour site and eradicate endothelial cells. The persistent lack of oxygen and nourishment starve, and eventually kill the tumour cells.

In another preferred embodiment the prodrug is ganciclovir [see Martin L A et al.: Direct cell killing by suicide genes; Cancer Metastasis Rev. 1996 15 301-316], and the prodrug metabolising gene is the herpes simplex virus thymidine kinase (HSV-tk) gene. Upon administration of the non-toxic prodrug ganciclovir, the thymidine kinase expressed by the endothelial cells is capable of metabolising ganciclovir to a toxic triphosphate form, which kills the endothelial cells.

In yet another preferred embodiment the prodrug is 6-thioxanthine, and the prodrug metabolising gene is E. coli xanthine-guanine phosphoribosyltransferase (E. coli-xgpt) 6pt.

In a further preferred embodiment the prodrug is 5-fluorocytosine [see Martin L A et al.: Direct cell killing by suicide genes; Cancer Metastasis Rev. 1996 15 301-316], and the prodrug metabolising gene is cytosine deamidase. Upon administration of the non-toxic prodrug 5-fluorocytosine, the cytosine deaminase expressed by the endothelial cells is capable of metabolising 5-fluorocytosine to 5-fluorouracil, which happens to be toxic to the endothelial cells.

The present invention further encompasses tissue-specific targeting the endothelial cells by the use of a cell specific promoter.

The viral vector system (gene transfer system) of the invention may be prepared by methods known by those skilled in the art, e.g. as described in Current Protocols in Molecular Biology [Ausubel et al. (Eds.): Current Protocols in Molecular Biology, Wiley and Sons Inc.].

Selective gene transfer and expression in endothelial cells has been obtained by transcriptional targeting whereby tissue-specific transcriptional regulatory elements (promoters) are used to limit the expression of the suicide gene to endothelial cells that express transcription factors that interact with those regulatory elements.

In a preferred embodiment the promoter is a vitronectin receptor α subunit (αv) gene promoter [see Donahue J P et al.: The integrin αv gene: identification and characterisation of the promoter region; Biochemica and Biophysica Acta 1994 1219 228-232]. The vitronectin receptor α subunit (αv) associates with receptors that are variously expressed by endothelial cells. The expression of the integrin αv accompanies angiogenesis. During angiogenesis αv expression is increased on the invasive endothelium. According to the present invention in vivo application of the adenoviral or replication-defective retrovirus vector under the control of the αv promoter induces high levels of the gene product primarily in the proliferating endothelial cells implicated in the angiogenic process.

In another preferred embodiment the promoter is a vascular endothelial growth factor (VEGF) receptor gene promoter. The VEGF receptor is up-regulated in tumour-induced proliferating endothelial cells. According to the present invention in vivo application of the adenoviral or replication-defective retrovirus vector under the control of the promoter for the VEGF receptor gene induces high levels of the gene product in primarily the proliferating endothelial cells implicated in the angiogenic process.

In a third preferred embodiment the promoter is a β33 integrin gene promoter. The β33 integrin is up-regulated in proliferating and invasive endothelial cells. According to the present invention in vivo application of the adenoviral or replication-defective retrovirus vector under the control of the promoter for the β3 subunit gene of the β33 integrin induces high levels of the gene product in primarily the proliferating invasive endothelial cells implicated in the angiogenic process.

In a fourth preferred embodiment the promoter is a vascular endothelial cadherin (VE-cadherin) gene promoter. The VE-cadherin integrin up-regulated in proliferating endothelial cells. According to the present invention in vivo application of the adenoviral or replication-defective retrovirus vector under the control of the promoter for the VE-cadherin gene induces high levels of the gene product in primarily the endothelial cells implicated in the angiogenic process.

At the onset of angiogenesis local expression of Angiopoietin 2 occurs in endothelial cells. In a fifth preferred embodiment the promoter is the Angiopoietin 2 gene promoter. Angiopoietin 2 is up-regulated in proliferating endothelial cells. According to the present invention in vivo application of the adenoviral or replication-efective retrovirus vector under the control of the promoter for the Angiopoietin 2 gene induces high levels of the gene product in primarily the endothelial cells implicated in the angiogenic process.

Recombinant Viral Vectors

In another aspect the invention provides a recombinant viral vector comprising, in an operable combination,

a promoter and a prodrug metabolising gene,

wherein the promoter is capable of selectively promoting the transcription (expression) of the prodrug metabolising gene in an endothelial cell.

Viruses useful as gene transfer vectors include papovavirus, adenovirus, vaccinia virus, adeno-associated virus, herpesvirus, and retroviruses.

Retroviruses consist of a protein envelope that surrounds core proteins and RNA. The RNA encodes two long terminal repeats (LTRs), which include promoter and enhancer regions flanking the genome, transcriptional regulatory signals including the CAP site and polyadenylation signals, and structural genes including the env gene (encoding the envelope proteins), the gag gene (encoding the viral core proteins), and the pol gene (encoding the reverse transcriptase), as well as the packaging signal, psi.

For use in human patients, the retroviral vectors must be replication defective. This prevents further generation of infectious retroviral particles in the target tissue. Instead the replication defective vector becomes a“captive” transgene stable incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Preferred viral vectors of the invention are adenovirus vectors, adeno-associated virus vectors, and replication-defective retrovirus vectors.

Suitable vectors include, but are not limited to, herpes simplex viral based vectors such as pHSV1 [Geller et al., Proc. Natl. Acad. Sci. U.S.A. 1990 87 8950-8954]; retroviral vectors such as MFG [Jaffee et al., Cancer Res. 1993 53 2221-2226], and in particular Moloney retroviral vectors such as LN, LNSX, LNCX, LXSN [Miller and Rosman, Biotechniques 1989 7 980-989] and semliki forest virus (“SFV”) vectors; vaccinia viral vectors such as MVA [Sutter and Moss, Proc. Natl. Acad. Sci. U.S.A. 1992 89 10847-10851]; adenovirus vectors such as pJM17 [Ali et al., Gene Therapy 1994 1 367-384; Berker, Biotechniques 1988 6 616-624; Wand and Finer, Nature Medicine 1996 2 714-716]; adeno-associated virus vectors such as AAV/neo [Mura-Cacho et al., J. Immunother. 1992 11 231-237]; lentivirus vectors [Zufferey et al., Nature Biotechnology 1997 15 871-875]; pET 11a, pET3a, pET11d, pET3d, pET22d, and pET12a (available from Novagen); plasmid AH5 (which contains the SV40 origin and the adenovirus major late promoter); pRC/CMV (available from In Vitrogen); pCMU II [Paabo et al., EMBO J. 1986 5 1921-1927]; pZipNeo SV [Cepko et al., Cell 1984 37 1053-1062]; pSR-alpha. (available from DNAX, Palo Alto, Calif.); pBK-CMV (available from Stratagene, La Jolla, Calif.); pCDNA3 (available from In Vitrogen, Carlsbad, Calif.); and pCDNA1 (available from In Vitrogen, Carlsbad, Calif.).

In a preferred embodiment the promoter is a vitronectin receptor α subunit (αv) gene promoter.

In another preferred embodiment the promoter is a vascular endothelial growth factor (VEGF) receptor gene promoter.

In a third preferred embodiment the promoter is a β3 integrin gene promoter.

In a fourth preferred embodiment the promoter is a vascular endothelial cadherin (VE-cadherin) gene promoter.

In a fifth preferred embodiment the promoter is an Angiopoietin 2 gene promoter.

In a preferred embodiment the prodrug is ifosfamide, and the prodrug metabolising gene is CYP 2B1.

In another preferred embodiment the prodrug is ganciclovir, and the prodrug metabolising gene is the herpes simplex virus thymidine kinase (HSV-tk) gene.

In yet another preferred embodiment the prodrug is 6-thioxanthine, and the prodrug metabolising gene is E. coli xanthine-guanine phosphoribosyltransferase (E. coli-xgpt) 6pt.

In a further preferred embodiment the prodrug is 5-fluorocytosine, and the prodrug metabolising gene is cytosine deamidase.

Methods of Inhibiting Angiogenesis

In a third aspect the invention provides a method of inhibiting angiogenesis of a tumour in a subject, which method comprises the subsequent steps of

(i) introducing to said subject a recombinant viral vector capable of transducing endothelial cells,

which vector comprises, in an operable combination,

(a) a prodrug metabolising gene, i.e. a nucleotide sequence encoding an enzyme that promotes the conversion of a prodrug into a (cytotoxic) drug; and

(b) a promoter capable of selectively promoting the transcription (expression) of the prodrug metabolising gene in an endothelial cell; and

(ii) introducing said prodrug to said subject;

wherein said endothelial cell is more sensitive to said (cytotoxic) drug than to said prodrug.

The viral vector may in particular be an adenovirus vector, an adeno-associated virus vector, or a replication-defective retrovirus vector.

In a preferred embodiment the promoter is a vitronectin receptor α subunit (αv) gene promoter.

In another preferred embodiment the promoter is a vascular endothelial growth factor (VEGF) receptor gene promoter.

In a third preferred embodiment the promoter is a β3 integrin gene promoter.

In a fourth preferred embodiment the promoter is a vascular endothelial cadherin (VE-cadherin) gene promoter.,

In a fifth preferred embodiment the promoter is an Angiopoietin 2 gene promoter

In a preferred embodiment the prodrug is ifosfamide, and the prodrug metabolising gene is CYP 2B1.

In another preferred embodiment the prodrug is ganciclovir, and the prodrug metabolising gene is the herpes simplex virus thymidine kinase (HSV-tk) gene.

In yet another preferred embodiment the prodrug is 6-thioxanthine, and the prodrug metabolising gene is E. coli xanthine-guanine phosphoribosyltransferase (E. coli-xgpt) 6pt.

In a further preferred embodiment the prodrug is 5-fluorocytosine, and the prodrug metabolising gene is cytosine deamidase.

Method of Therapy

In another aspect the invention provides a method for the treatment or alleviation of diseases or disorders or conditions of living animal bodies, including humans, which diseases, disorders or conditions are responsive to anti-angiogenesis.

In a more preferred embodiment the invention provides a method of genetic pro-drug activation therapy of treating diseases or disorders or conditions of a living animal body, including a human, which diseases or disorders or conditions is responsive to the inhibition of proliferating endothelial cells, such as, but not limited to, solid tumour growth, metastases, diabetic retinopathy, psoriasis, macular degeneration, and inflammation-related diseases such as rheumatoid arthritis, ulcerative colitis and osteoarthritis.

### BRIEF DESCRIPTION OF THE DRAWINGS

The present invention is further illustrated by reference to the accompanying drawing, in which:

FIG. 1A shows a map of plasmid pc3/2B1 harbouring the CYP2B1 gene;

FIG. 1B shows a map of vector pCMVmAB harbouring the CMV promoter; and

FIG. 1C shows a map of virus vector pCMVcyp2B1 harbouring the CYP2B1 gene and the CMV promoter.

### EXAMPLE

The invention is further illustrated with reference to the following example, which is not intended to be in any way limiting to the scope of the invention as claimed.

This example describes an in vitro study designed to demonstrate proof of principle of the method of the invention. The study is carried out using human umbilical vein epithelial (HUVEC) cells. In this example the prodrug is ifosfamide, and the prodrug metabolising gene is cytochrome P450 2B1 (CYP2B1) gene.

The first step describes the construction, test and validation of an adenoviral vector expressing the CYP2B1 gene for the purpose of adenoviral mediated gene transfer, and the second step describes how the effect on proliferation (bio activity) can be analysed.

Construction, Test and Validation of an Adenoviral CYP2B1 Gene

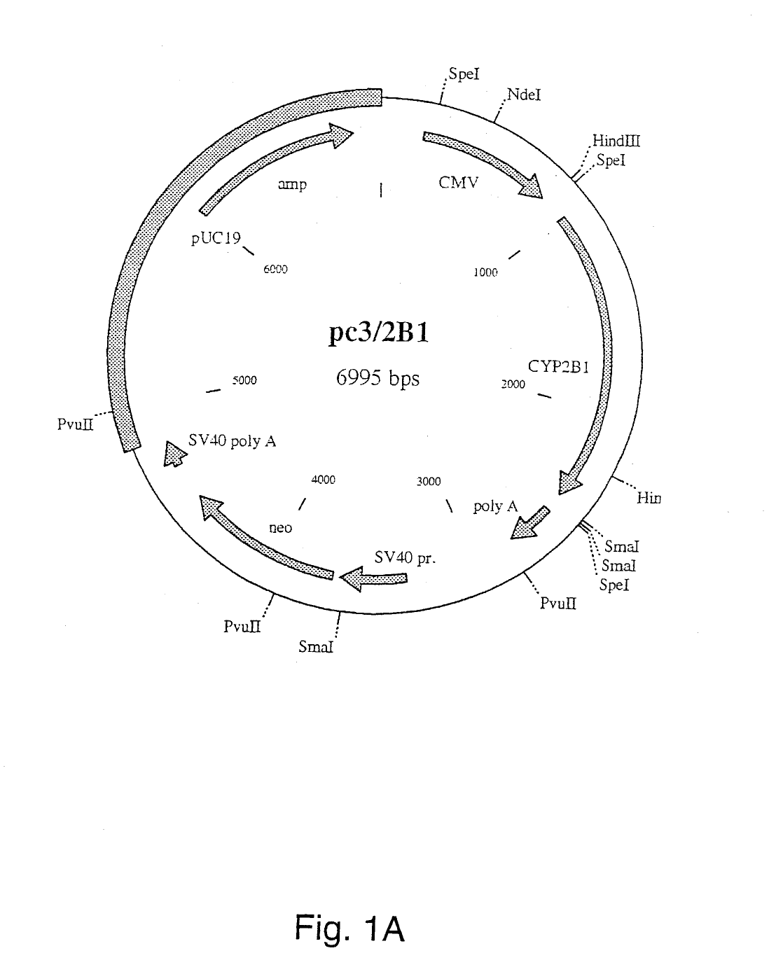

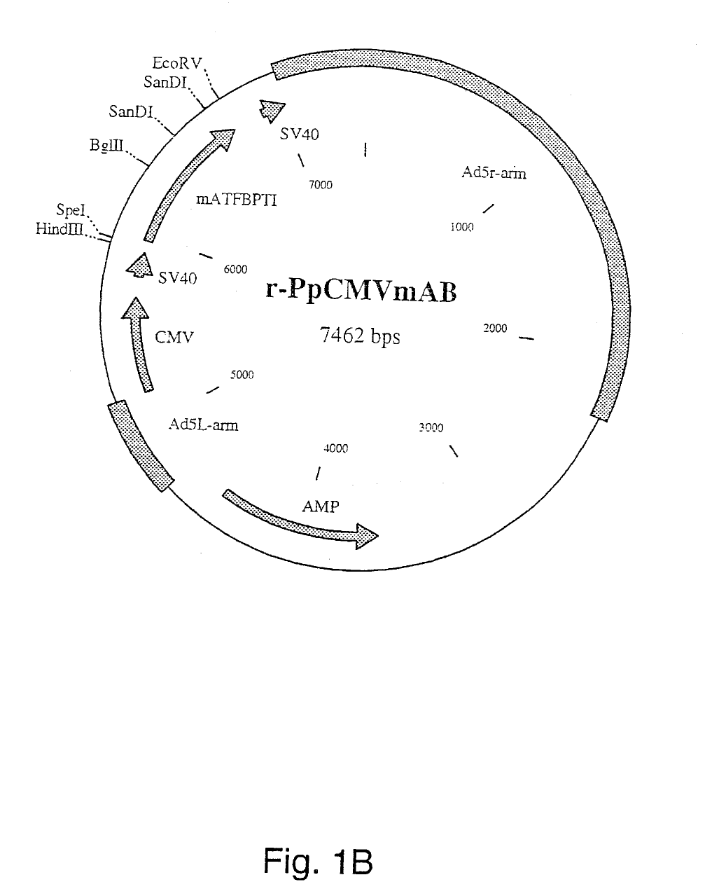

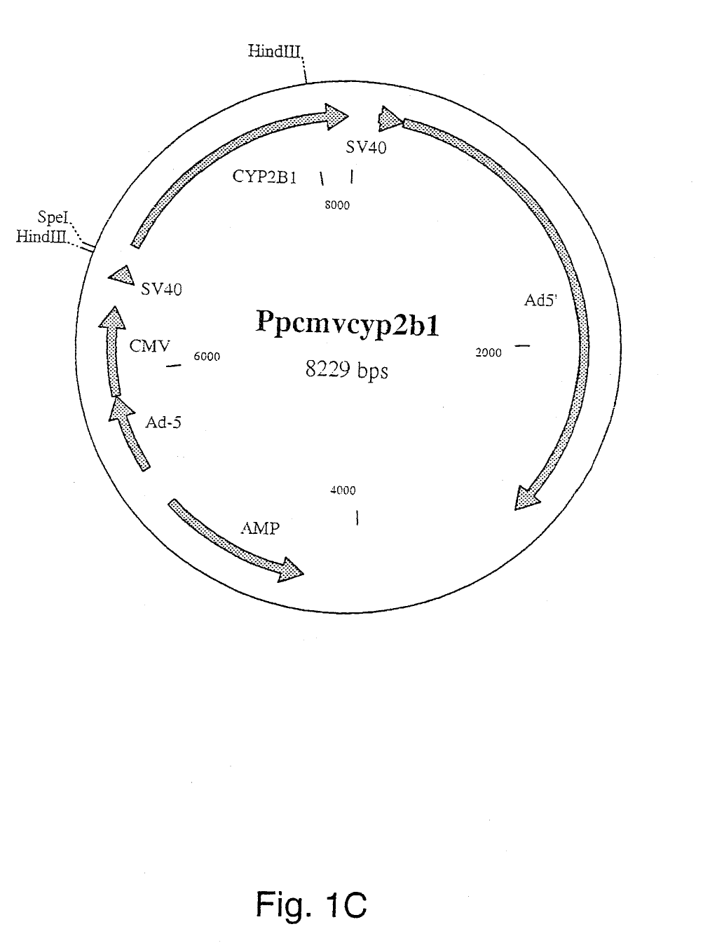

Plasmid pc3/2B1 harbouring the CYP2B1 gene (cf. FIG. 1A) was digested with restriction enzymes Spel and Smal, and the 1584 bps 2B1 fragment was purified from an agarose gel. Vector pCMVmAB harbouring the CMV promoter (cf. FIG. 1B) was digested with restriction enzymes SpeI and EcoRV, and the 6645 bps linear pCMV10 fragment was purified from an agarose gel. The vector and the CYP2B1 insert from plasmid pc3/2B1 was ligated in molar ratios from 1:1 to 1:3 and 1:5 to give the virus vector pCMVcyp2B1 harbouring the CYP2B1 and the CMV promoter and adenoviral structural genes (jf. FIG. 1C).

As an alternative route, in order to increase the success rate by minimising the number of purification steps, we performed extra digestions of pc3/2B1 with PVUII and Ndel to destroy the backbone and interfering blunt/Spel fragments, and extra digestions of PpCMVmAB with BgIII and SanDI to destroy the Spel/EcoRV mAB insert. This DNA-fragment mixture was purified using purification columns and ligated at the same ratios as mentioned above.

10 cm² wells containing human embryonic retinoblast cells (HER 911 cells) [Watzlik et al., Gene Therapy 2000 7 (1) 70-74] at a cell density of approximately 200,000 cells/cm² were transfected with 0,8 μg of the shuttle vector pJM17 [Ali et a., Gene Therapy 1994 1 367-384; Berker, Biotechniques 1988 6 616-624; Wand and Finer, Nature Medicine 1996 2 714-716] and 0,1/0,2/0,4 μg of PVUI linearized PpCMVcyp2B1, using transfecting agent Fugene6 (available from Roche).

Ten days after transfection, eight recombinant plaques appeared on cells transfected with the clone. These plaques were picked and screened for the CYP2B1 insert by PCR using the following universal primers for the adenovirus 5 vector harbouring the CMV promoter (Ad₅CMV):

Cmv-forward: 5′-aactgctcctcagtggatgttg-3′; and

Cmv-reverse: 5′-tctagcagcacgccatagtgac-3′.

A plaque (Ad₅CMV-C2B1) was amplified on 10 cm² wells, in 75 cm² tissue culture flasks and finally in 5×500 cm² tissue culture flasks.

The Adenoviruses were purified from the cell lysates of these final amplification steps using caesiumchloride density gradient ultracentrifugation steps followed by dialysis.

Viral stocks were aliquoted and stored at −80° C.

A viral titer of 4×10¹⁰ PFU/ml was determined using HER 911 cells and an agarose overlay assay.

Recombinant viral genomes from all steps were screened for the CYP2B1 insert by PCR using universal primers for Ad₅CMV.

To test transgene expression, cell lysates from the virus producing cells were submitted to Western blot analysis.

Functional Bio-assay

To test the bioactivity of the CYP2B1 transgene, human umbilical vein epithelial (HUVEC) cells [Jaffe et al.; J. Clin. Invest. 1973 52 2745-2746] were infected with both viral clones and treated with the prodrug Holoxan (Ifosfamide) according to the following protocol.

HUVEC cells were infected with Ad5C2B1 on day 2 with concentrations ranging from 0 to 2×10⁸ PFU/ml for 2 hours.

The cells were seeded at 40.000 cells/well at day 3 (some wells were not sub-cultured but saved for cell extracts and medium collection at day 4).

At day 4 Ifosfamide was added at concentrations ranging of from zero to 0,5 mM. HUVEC cells were grown on gelatin-coated tissue culture dishes in medium 199 (Biowhitakker), supplemented with 10% (vol/vol) human serum, 10% (v/v) heat-inactivated newborn calf serum (Biowhitakker), 5 U/mL of heparin (Leo Pharma BV), 150 mg/mL of crude EC growth factor (TNO-PG, Leiden), 100 U/mL of penicillin, and 100 mg/mL of streptomycin (Biowhitakker). Cells were grown at 37° C. in a humid atmosphere with 5% (vol/vol) CO₂.

Cell numbers were determined at day 8 using crystal-violet staining and optical density measurement.

To quantify the effect off viral infection and prodrug treatment the cells numbers were determined as described above.

The results are presented in Table 1 below, and show an inhibition of approximately 80% in CYP2B1 transfected endothelial cells treated with a maximum concentration of 0.5 nM Ifosfamide compared with non-transfected cell treated with the vehicle.

TABLE 1
Endothelial Cell Proliferation
Cell Number as a result of Virus Concentration (transfection) [0,
1.25 × 10⁷, 2.5 × 10⁷, 5 × 10⁷, 1 × 10⁸ and 2 × 10⁸ PFU/ml]
and Prodrug (Ifosfamide) Concentration [0, 0.0625, 0.125, 0.25 and
0.5 mM]

| Number of | | | | | |
|---|---|---|---|---|---|
| Cells | | | | | |
| Virus | | | | | |
| Concentration | Ifosfamide Concentration (mM) | | | | |
| (PFU/ml) | 0 | 0.0625 | 0.125 | 0.25 | 0.50 |
| 0 | 140000 | 152000 | 151600 | 152500 | 116000 |
| 1.25 × 10⁷ | 110000 | 98000 | 110000 | 102000 | 85000 |
| 2.5 × 10⁷ | 80000 | 88000 | 85000 | 79200 | 56000 |
| 5 × 10⁷ | 94000 | 87000 | 85000 | 75000 | 55000 |
| 1 × 10⁸ | 78000 | 80500 | 63600 | 66000 | 38500 |
| 2 × 10⁸ | 73000 | 60000 | 54000 | 53000 | 30000 |

1. A method of sensitising an endothelial cell to a prodrug, which method comprises the steps of
(i) transfecting said endothelial cell with a polynucleotide that comprises, in an operable combination,
(a) a prodrug metabolising gene, i.e. a nucleotide sequence encoding an enzyme that promotes the conversion of said prodrug into a (cytotoxic) drug; and
(b) a promoter capable of selectively promoting the transcription (expression) of the prodrug metabolising gene in said endothelial cell; and
(ii) delivering said prodrug to said endothelial cell;
wherein said endothelial cell is more sensitive to said (cytotoxic) drug than to said prodrug.

2. The method according to claim 1, wherein said promoter is
a vitronectin receptor a subunit (αv) gene promoter;
a vascular endothelial growth factor (VEGF) receptor gene promoter;
a β3 integrin gene promoter;
a vascular endothelial cadherin (VE-cadherin) gene promoter; or
an angiopoietin 2 gene promoter.

3. The method according to either of claims 1-2, wherein
the prodrug is ifosfamide, and the prodrug metabolising gene is CYP 2B1;
the prodrug is ganciclovir, and the prodrug metabolising gene is the herpes simplex virus thymidine kinase (HSV-tk) gene;
the prodrug is 6-thioxanthine, and the prodrug metabolising gene is E. coli xanthine-guanine phosphoribosyltransferase (E. coli-xgpt) 6pt; or
the prodrug is 5-fluorocytosine, and the prodrug metabolising gene is cytosine deamidase.

4. A recombinant viral vector comprising, in an operable combination,
a promoter and a prodrug metabolising gene,
wherein the promoter is capable of selectively promoting the transcription (expression) of the prodrug metabolising gene in an endothelial cell.

5. The recombinant viral vector according to claim 4, wherein the promoter is
a vitronectin receptor α subunit (αv) gene promoter;
a vascular endothelial growth factor (VEGF) receptor gene promoter;
a β3 integrin gene promoter;
a vascular endothelial cadherin (VE-cadherin) gene promoter; or
an angiopoietin 2 gene promoter.

6. The recombinant viral vector according to claim 4, wherein
the prodrug is ifosfamide, and the prodrug metabolising gene is CYP 2B1;
the prodrug is ganciclovir, and the prodrug metabolising gene is the herpes simplex virus thymidine kinase (HSV-tk) gene;
the prodrug is 6-thioxanthine, and the prodrug metabolising gene is E. coli xanthine-guanine phosphoribosyltransferase (E. coli-xgpt) 6pt; or
the prodrug is 5-fluorocytosine, and the prodrug metabolising gene is cytosine deamidase.

7. The recombinant viral vector according to any of claims 4-6, the viral vector being an adenovirus vector, an adeno-associated virus vector, or a replication-defective retrovirus vector.

8. A method of inhibiting angiogenesis of a tumour in a subject, which method comprises the subsequent steps of
(i) introducing to said subject a recombinant viral vector capable of transducing endothelial cells,
which vector comprises, in an operable combination,
(a) a prodrug metabolising gene, i.e. a nucleotide sequence encoding an enzyme that promotes the conversion of a prodrug into a (cytotoxic) drug; and
(b) a promoter capable of selectively promoting the transcription (expression) of the prodrug metabolising gene in an endothelial cell; and
(ii) introducing said prodrug to said subject;
wherein said endothelial cell is more sensitive to said (cytotoxic) drug than to said prodrug.

9. The method of claim 8, wherein the promoter is
a vitronectin receptor a subunit (αv) gene promoter;
a vascular endothelial growth factor (VEGF) receptor gene promoter;
a β3 integrin gene promoter;
a vascular endothelial cadherin (VE-cadherin) gene promoter; or
an angiopoietin 2 gene promoter.

10. The method of claim 8, wherein
the prodrug is ifosfamide, and the prodrug metabolising gene is CYP 2B1;
the prodrug is ganciclovir, and the prodrug metabolising gene is the herpes simplex virus thymidine kinase (HSV-tk) gene;
the prodrug is 6-thioxanthine, and the prodrug metabolising gene is E. coli xanthine-guanine phosphoribosyltransferase (E. coli-xgpt) 6pt; or
the prodrug is 5-fluorocytosine, and the prodrug metabolising gene is cytosine deamidase.

11. The method according to any of claims 8-10, wherein the viral vector is an adenovirus vector, an adeno-associated virus vector, or a replication-defective retrovirus vector.
